(19) United States
(12) Patent Application Publication
(10) Pub. No.: US 2025/0288241 A1
(43) Pub. Date: Sep. 18, 2025

(54) SUPPORT DEVICE, SUPPORT SYSTEM, AND PROGRAM FOR ELECTROCARDIOGRAM ANALYSIS

(71) Applicant: Cardio Intelligence Inc., Tokyo (JP)

(72) Inventors: Tomohiro TAKATA, Tokyo (JP); Mineki TAKECHI, Tokyo (JP)

(21) Appl. No.: 18/789,019

(22) Filed: Jul. 30, 2024

Related U.S. Application Data
(63) Continuation of application No. PCT/JP2024/022966, filed on Jun. 25, 2024.

(30) Foreign Application Priority Data
Mar. 13, 2024 (JP) 2024-039336

(51) Int. Cl. A61B 5/366; A61B 5/339

(52) U.S. Cl. CPC A61B 5/366; A61B 5/339

(57) ABSTRACT

The support system includes an electrocardiograph, an electrocardiogram analyzer, and a support device. The electrocardiograph is configured to acquire an electrocardiogram of a subject. The electrocardiogram analyzer is configured to receive the electrocardiogram, to divide the electrocardiogram into a plurality of sections, and to extract the sections other than specified sections from the plurality of sections as candidate sections. The support device is configured to be communicatively connected to the electrocardiogram analyzer. The support device includes a computing device and a display device controlled by the computing device. The functions of the computing device are described in the specification in detail.

### CROSS-REFERENCE TO RELATED APPLICATIONS

This application is a Continuation of International Patent Application No. PCT/JP2024/022966, filed on Jun. 25, 2024, which claims the benefit of priority to Japanese Patent Application No. 2024-039336, filed on Mar. 13, 2024, the entire contents of which are incorporated herein by reference.

### FIELD

An embodiment of the present invention relates to a support device and a support system for electrocardiogram analysis as well as a method for analyzing electrocardiograms. Alternatively, an embodiment of the present invention relates to a program for supporting electrocardiogram analysis.

### BACKGROUND

Electrocardiograms are widely used as a means for determining the health status of patients. However, some types of diseases require measurement over a long time period (e.g., one day), and even if long-time measurement is carried out, waveforms caused by diseases may not clearly appear in electrocardiograms. Hence, systems have been developed in recent years to automatically determine whether or not there are signs of cardiac diseases in electrocardiograms and to automatically determine the probability of their occurrence and so on through analysis using supervised learning models (see Japanese Patents No. 6,865,329 and 7,002,168).

### SUMMARY

An embodiment of the present invention is a support system for electrocardiogram analysis. The support system includes an electrocardiograph, an electrocardiogram analyzer, and a support device. The electrocardiograph is configured to acquire an electrocardiogram of a subject. The electrocardiogram analyzer is configured to receive the electrocardiogram, to divide the electrocardiogram into a plurality of sections, and to extract the sections other than specified sections from the plurality of sections as candidate sections. The support device is configured to be communicatively connected to the electrocardiogram analyzer. The support device includes a computing device and a display device controlled by the computing device. The computing device is configured to execute (1) accepting a first user command to upload the electrocardiogram to the electrocardiogram analyzer, (2) displaying the candidate sections on a screen of the display device, (3) accepting a second user command to select a non-analysis section from the candidate sections, and (4) transmitting an instruction to the electrocardiogram analyzer to analyze the electrocardiogram using analysis sections which are not selected by the second user command among the candidate sections. The specified sections include sections indicating a waveform caused by a disease.

An embodiment of the present invention is a support device for electrocardiogram analysis. The support device for the electrocardiogram analysis includes a computing device and a display device controlled by the computing device. The computing device is configured to execute (1) accepting a first user command to cause the computing device to upload an electrocardiogram of a subject to an electrocardiogram analyzer communicatively connected to the computing device and configured to divide the electrocardiogram into a plurality of sections and extract the sections other than specified sections from the plurality of sections as candidate sections, (2) displaying the candidate sections on a screen of the display device, (3) accepting an input of a second user command for selecting a non-analysis section from the candidate sections, and (4) transmitting, to the electrocardiogram analyzer, a command for analyzing the electrocardiogram of the subject using analysis sections which are not selected from the candidate sections by the second user command. The specified sections include the sections exhibiting a waveform caused by a disease.

An embodiment of the present invention is a program for supporting electrocardiogram analysis performed on an electrocardiogram analyzer using a computing device. The electrocardiogram analyzer is communicatively connected to the computing device and is configured to divide an electrocardiogram of a subject into a plurality of sections and extract the sections other than specified sections from the plurality of sections as candidate sections. The program is configured to cause the computing device to execute (1) accepting a first user command to upload the electrocardiogram of the subject to the electrocardiogram analyzer, (2) displaying the candidate sections on a screen of the display device of the computing device, (3) accepting a second user command for selecting a non-analysis section from the candidate sections, and (4) transmitting, to the electrocardiogram analyzer, a command for analyzing the electrocardiogram using analysis sections which are not selected from the candidate sections by the second user command.

An embodiment of the present invention is a computer-readable storage medium in which the aforementioned program is recorded.

An embodiment of the present invention is an analysis method of an electrocardiogram. The analysis method includes (1) acquiring an electrocardiogram of a subject, (2) dividing the electrocardiogram into a plurality of sections, (3) extracting the sections other than specified sections from the plurality of sections as candidate sections, (4) selecting a non-analysis section from the candidate sections, and (5) analyzing the electrocardiogram using analysis sections which are not selected from the candidate sections as the non-analysis section.

### BRIEF DESCRIPTION OF DRAWINGS

FIG. 1 is a block diagram of a support system for electrocardiogram analysis according to an embodiment of the present invention.

FIG. 2 is a flow of electrocardiogram analysis using a support system for electrocardiogram analysis according to an embodiment of the present invention.

FIG. 3 is a schematic view for explaining an operation performed by a support system for electrocardiogram analysis according to an embodiment of the present invention.

FIG. 4 is an example of a display displayed on a display device of a support device according to an embodiment of the present invention.

FIG. 5 is an example of a display displayed on a display device of a support device according to an embodiment of the present invention.

FIG. 6 is an example of a display displayed on a display device of a support device according to an embodiment of the present invention.

FIG. 7 is an example of a display displayed on a display device of a support device according to an embodiment of the present invention.

FIG. 8 is an example of a display displayed on a display device of a support device according to an embodiment of the present invention.

FIG. 9 is an example of a display displayed on a display device of a support device according to an embodiment of the present invention.

FIG. 10 is an example of a display displayed on a display device of a support device according to an embodiment of the present invention.

FIG. 11 is an example of a display displayed on a display device of a support device according to an embodiment of the present invention.

### DESCRIPTIONS OF EMBODIMENTS

Hereinafter, each embodiment of the present invention is explained with reference to the drawings. The invention can be implemented in a variety of different modes within its concept and should not be interpreted only within the disclosure of the embodiments exemplified below.

The drawings may be illustrated so that the width, thickness, shape, and the like are illustrated more schematically compared with those of the actual modes in order to provide a clearer explanation. However, they are only an example, and do not limit the interpretation of the invention. In the specification and each drawing, the same reference number is provided to an element that is the same as that which appears in preceding drawings, and a detailed explanation may be omitted.

### 1. Configuration of Support System for Electrocardiogram Analysis

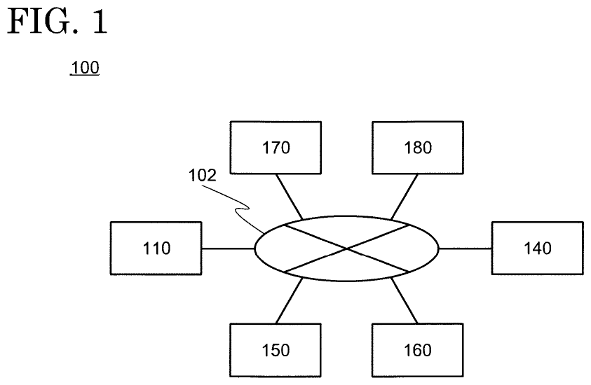

A block diagram of a support system 100 for electrocardiogram analysis according to an embodiment of the present invention is shown in FIG. 1. As shown in FIG. 1, the support system 100 includes a support device 110, an electrocardiogram analyzer 140, and an electrocardiograph 150. The support system 100 may further include a database 160. There is no restriction on the number of electrocardiographs 150, electrocardiogram analyzers 140, and support devices 110 included in the support system 100, and a plurality of electrocardiographs 150, a plurality of support devices 110, and a plurality of databases 160 may be included, for example. The support system 100 may also include a plurality of electrocardiogram analyzers 140. The support devices 110 are controlled by a program (described below) which operates according to user commands. The support system 100 may further include a heart rate monitor 170 for measuring a user's heart rate, an accelerometer 180 for detecting user's conditions, and the like.

The support device 110 and the electrocardiogram analyzer 140 are communicatively connected to each other by a network 102. An example of the network 102 is a wide-area network such as the Internet. When the support system 100 includes the database 160, the database 160 may also be communicatively connected to the support device 110 and the electrocardiogram analyzer 140 via the network 102. The electrocardiograph 150 may also be communicatively connected by the network 102 to the support device 110, the electrocardiogram analyzer 140, and/or the database 160, or it may be configured not to communicate via the network 102. In the latter case, the electrocardiogram data acquired by the electrocardiograph 150 may be stored in the support device 110 and/or the database 160 via a storage medium such as a flash memory.

The electrocardiograph 150 is a device for acquiring an electrocardiogram of a subject, and any known electrocardiograph may be used as the electrocardiograph 150. When the electrocardiogram is acquired over a long time period, a Holter electrocardiograph or an event recorder, which are less burdensome on the subject, may be used as the electrocardiograph 150. The electrocardiograph 150 acquires the potential changes caused by the flow of electricity in the heart and provides an electrocardiogram as a plot of the potential changes versus measurement time.

The electrocardiogram analyzer 140 is a device for analyzing the electrocardiogram data acquired by the electrocardiograph 150. The electrocardiogram analyzer 140 is a device with computational and communication functions and may be a personal computer or an application server. As described below with respect to the functions and operation of the electrocardiogram analyzer 140, it is possible to determine whether the subject has diseases or test abnormalities on the basis of the analysis results obtained by the electrocardiogram analyzer 140. The diseases and test abnormalities which can be detected by the electrocardiogram analyzer 140 are not limited to cardiac diseases, and the electrocardiogram analyzer 140 can detect a variety of diseases and test abnormalities. For example, the electrocardiogram analyzer 140 is capable of detecting, from the electrocardiogram data of the subject, not only diseases and test abnormalities of the heart system such as atrial fibrillation, heart failure, ventricular arrhythmia, valvular disease, cardiomyopathy, and the like, but also test abnormalities indicating the possibility of various diseases such as hyponatremia, diabetes, and hyperkalemia.

The support device 110 is a terminal used by the user such as a physician and other medical professional. Although not illustrated, the support device 110 has a computing device and a display device controlled by the computing device. The computing device is a device having computing and communication functions and may be, for example, a desktop-type personal computer or a portable communication terminal such as a smartphone and a tablet. The computing device is connected to or incorporates an input interface such as a keyboard, a mouse, and a touch panel, and the user uses these input interfaces to input user commands to the computing device. The display device is connected to or incorporated into the computing device and serves as an interface to the user. Details of the functions and operation of the support device 110 are also described below.

The database 160 may be a storage device connected to or mounted on a communication-computing terminal such as a personal computer or may be a so-called file server specialized for storing and managing electronic data.

The heart rate monitor 170 and the accelerometer 180 are attached to the subject, which allows the state of the subject (e.g., the subject is sleeping, at rest, or exercising including walking or running) to be determined. The heart rate monitor 170 and the accelerometer 180 may also be connected to the electrocardiogram analyzer 140 via the network 102. The information from the heart rate monitor 170 and the accelerometer 180 may be linked to the electrocardiogram data in the electrocardiogram analyzer 140 and may be stored together with the electrocardiogram data in the electrocardiogram analyzer 140 and/or the database 160.

### 2. Electrocardiogram Analysis

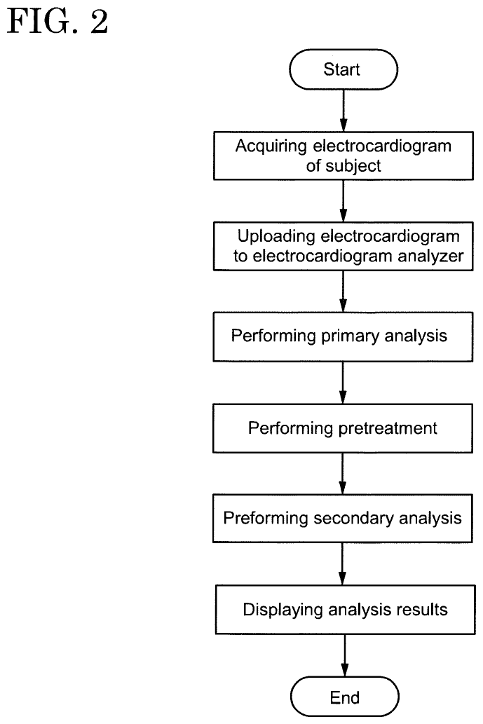

The following describes the electrocardiogram analysis using the support system 100 along with the functions of the support device 110 and the electrocardiogram analyzer 140. A flowchart of the electrocardiogram analysis is shown in FIG. 2. In this electrocardiogram analysis, after acquiring the subject's electrocardiogram, two analyses (primary analysis and secondary analysis) using a supervised learning model are performed, and pretreatment for the secondary analysis is further performed between the primary analysis and the secondary analysis. The details are described below.

### (1) Acquiring Electrocardiogram

First, an electrocardiogram of a subject is acquired using the electrocardiograph 150. The electrocardiogram measurement time may be, for example, equal to or longer than 10 seconds, equal to or longer than 1 hour, equal to or longer than 12 hours, equal to or longer than 1 day, or equal to or longer than 3 days. Measurement for a long time period increases the probability of detecting disease or test abnormalities. There is also no limit to the maximum measurement time which may be 1 week, 3 days, 1 day, or 12 hours, for example. The acquired electrocardiograms are stored in the support device 110 and/or the database 160 via communication from the electrocardiograph 150 over the network 102 or via a storage medium. At the same time, information obtained from the heart rate monitor 170 and the accelerometer 180 may also be stored in the support device 110, the electrocardiogram analyzer 140, and/or the database 160 via communication over the network 102 or via a storage medium.

### (2) Primary Analysis

The electrocardiogram is subjected to the primary analysis by the electrocardiogram analyzer 140. Specifically, first, the support device 110 uploads the electrocardiogram data stored in the support device 110 and/or the database 160 to the electrocardiogram analyzer 140 via the network 102 according to a user command received via the input interface. In the case where the electrocardiogram data is stored in the electrocardiogram analyzer 140, this step need not be performed.

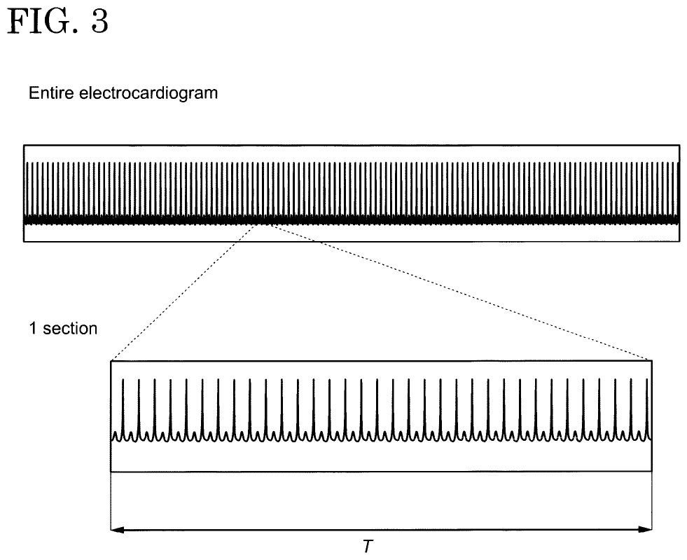

The electrocardiogram analyzer 140 divides the transmitted or stored electrocardiogram into a plurality of sections. The duration of each section may be set arbitrarily within a range shorter than the acquisition time of the electrocardiogram, which may be, for example, in a range equal to or longer than seconds and equal to or shorter than 120 seconds and is typically 30 seconds. As described above, since electrocardiograms are acquired over a relatively long period of time, a portion of one entire electrocardiogram is a single section, and a single electrocardiogram is composed of a plurality of sections as schematically shown in FIG. 3. For example, when an electrocardiogram acquired over a 24-hour measurement is divided into 30-second sections, a single electrocardiogram is composed of 2,880 sections.

Each section is then subjected to the analysis (primary analysis) by the electrocardiogram analyzer 140. In the primary analysis, the presence or absence of one or more diseases or test abnormalities is determined by using a supervised learning model. Specifically, a number of normal electrocardiograms, i.e., electrocardiograms with a number of humans having no diseases or test abnormalities listed by the user as test items, are acquired, each electrocardiogram is divided into a plurality of sections, and the data of each section is created as input data (features). Similarly, electrocardiograms of a number of humans with diseases or abnormalities listed by the user as test items are acquired, each electrocardiogram is divided into a plurality of sections, and the data of each section is created as input data. These input data are used to train the supervised learning model.

In the primary analysis, the electrocardiogram of the subject who is the subject of the examination is used and analyzed using the supervised learning model. The output data at this time are the judgment results for each section and include two categories of whether or not each section is a section in which a disease or test abnormality listed by the user as test items appears. As described above, there are no particular restrictions on the diseases or test abnormalities which are the detection targets, and input data for each of various diseases or test abnormalities in addition to heart diseases may be created to train the supervised learning model. Alternatively, the diseases or test abnormalities listed as test items in the primary analysis may be diseases or test abnormalities of the cardiac system.

Algorithms for the supervised learning model include linear regression models, random forests, decision trees, gradient boosting, and deep learning, and the deep learning which promises high accuracy is particularly preferred. When deep learning is used, convolutional neural networks, recurrent neural networks, Vision Transformer models, and the like may be used as appropriate.

If the primary analysis detects a section where the diseases or test abnormalities listed by the user as test items appear, the user may recheck the electrocardiogram, determine whether the results of the primary analysis are correct, and take appropriate action on the basis of the determination. However, primary analysis does not always accurately detect the sections where a disease or a test abnormality appears. In addition, a normal section may be erroneously detected as a section where a disease or a test abnormality appears. Furthermore, although the training model is trained using, as the input data, the electrocardiograms of humans with diseases or abnormalities listed by the user as test items and the electrocardiograms of normal humans who do not have these diseases or abnormalities as input data in the primary analysis, the signs thereof may not clearly appear on the electrocardiogram depending on the type of disease or test abnormalities. For example, even if the electrocardiogram of a subject with some disease or test abnormalities is acquired over a long time period, the electrocardiogram may not contain any sections exhibiting waveforms caused by the disease or test abnormalities, and the electrocardiogram may consist of sections which can be recognized as sections substantially showing sinus waveforms. In such cases, the primary analysis will miss those diseases or test abnormalities. Furthermore, it may be necessary to diagnose the presence or absence of a disease or test abnormality which is not listed as the test items in the primary analysis. For this reason, in the electrocardiogram analysis method according to an embodiment of the invention, the electrocardiogram data after the primary analysis is used for the secondary analysis described below.

### (3) Pretreatment

Unlike the primary analysis, the following two types of data are used in the preparation of the input data in the secondary analysis. One is the data of each section obtained by dividing the electrocardiogram data of a number of humans, who do not have the diseases or the test abnormalities listed by the user as test items, into a plurality of sections. On the other hand, the other electrocardiogram data is the data of the sections in which the disease is not manifested among the plurality of sections obtained by dividing the electrocardiogram data of a number of humans with the disease or the test abnormalities. For example, when the disease or the test abnormality is paroxysmal arrhythmia (paroxysmal atrial fibrillation, ventricular tachycardia, supraventricular tachycardia, and atrial flutter), the sections in which the waveform indicating the paroxysmal arrhythmia does not appear are used. In other words, the electrocardiogram data, which is difficult to use for the user to judge that a disease or a test abnormality exists from the subject's electrocardiogram, is used as one of the input data in the secondary analysis.

By using a supervised learning model trained using the aforementioned input data, the disease or the test abnormalities can be detected with high accuracy even when it is difficult for the user to determine the presence of the disease or the test abnormalities from the subject's electrocardiogram. For example, among analysis sections, a section indicating sinus rhythm, a section indicating extrasystole, a section indicating leg block, a section indicating QRS width variation, a section indicating decreased heart rate variability, and a section indicating T wave amplitude variation are used in the secondary analysis. Even if the analysis sections indicate sinus rhythm, the supervised learning model can detect diseases or test abnormalities such as atrial fibrillation, heart failure, and ventricular arrhythmia. When a section indicating extrasystole is included in the analysis sections, diseases or test abnormalities such as ventricular arrhythmia and valvular disease can be detected. On the other hand, when the analysis sections include a section indicating leg block, a section indicating QRS width variation, a section indicating decreased heart rate variability, and a section indicating T wave amplitude variation, diseases or test abnormalities such as cardiomyopathy, hyponatremia, diabetes mellitus, and hyperkalemia can be detected.

Therefore, it is difficult to accurately detect diseases or test abnormalities when the sections in which diseases or test abnormalities appear are used in the secondary analysis. Furthermore, there are sections in the electrocardiogram data which contain waveforms inappropriate for secondary analysis, such as a section including artifacts. Hence, in the electrocardiogram analysis method according to an embodiment of the present invention, prior to the secondary analysis, a pretreatment is performed to identify and exclude the sections (specified sections) which are inappropriate for the secondary analysis from the plurality of sections used in the primary analysis and to extract the remaining sections as candidate sections for the secondary analysis.

Specifically, the section in which the diseases or test abnormalities appeared in the primary analysis is identified as the specified section by the electrocardiogram analyzer 140. Furthermore, the electrocardiogram analyzer 140 may be configured to determine whether each section contains noise unrelated to the diseases or the test abnormalities and to identify the section containing the noise as the specified section. The noise includes AC noise caused by electrostatic induction, electromagnetic induction, and leakage current, electromyograms unrelated to the subject's disease, drift caused by baseline fluctuations, noise caused by static electricity, and the like. There are no restrictions on the method for determining the presence or absence of the noise, and any known method or algorithm may be applied. For example, a supervised learning model, which is machine-learned using noise-containing electrocardiograms and noise-free electrocardiograms as input data in advance, may be used to determine whether or not each of the sections of the subject's electrocardiogram contains noise. The section determined to be the specified section is excluded from the plurality of sections, and the remaining sections are extracted as candidate sections for the user's judgment on the support device 110 described below. For this purpose, linking is performed on each section in the pretreatment. For example, an identifier is provided to each section of the subject's electrocardiogram in chronological order to identify it, and a flag is provided to each identifier to identify that it has been extracted as the candidate section and/or that it has not been extracted (i.e., identified as the specified section).

The sections containing waveforms inappropriate for the secondary analysis are subsequently excluded. However, this process requires the knowledge of the user who is a medical specialist. For this reason, the support system 100 provides an opportunity to eliminate the sections inappropriate for the secondary analysis from the extracted candidate sections on the basis of the expert knowledge of the user. Specifically, the electrocardiogram analyzer 140 displays the plurality of candidate sections on the screen of the display device of the support system 110 in accordance with the instructions transmitted from the support device 110. Among the displayed candidate sections, the user selects the candidate section inappropriate for detecting diseases or test abnormalities in the secondary analysis, such as the candidate sections containing artifacts, and eliminates it from the candidate sections as a non-analysis section. This process allows the selection of appropriate candidate sections for the secondary analysis as analysis sections.

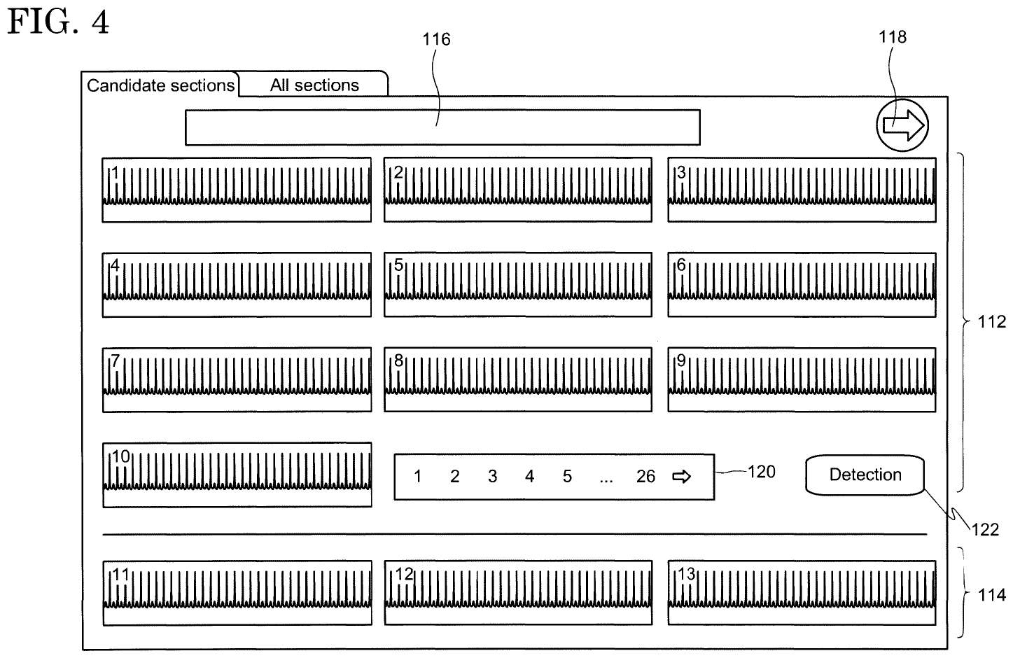

The display method at this time can be arbitrarily set. An example of the display on the screen of the display device of the support device 110 is shown in FIG. 4. In the example shown in FIG. 4, the first 10 candidate sections of the plurality of sections structuring the electrocardiogram are shown in an upper region 112 in chronological order. There is no restriction on the number (n) of candidate sections to be displayed at this time, and it may be changed as appropriate depending on the size of the display device and the like. Alternatively, the program may be configured to allow the user to set the number of candidate sections to be displayed. For example, 1 to 20 candidate sections, typically 10 candidate sections may be displayed on one screen. The candidate sections may be displayed on a single line or over a plurality of lines as shown in FIG. 4. Preferably, the candidate sections are displayed over a plurality of rows and a plurality of columns. Since the candidate sections can be easily compared with one another by displaying them in this manner, the sections inappropriate for the secondary analysis can be more efficiently detected.

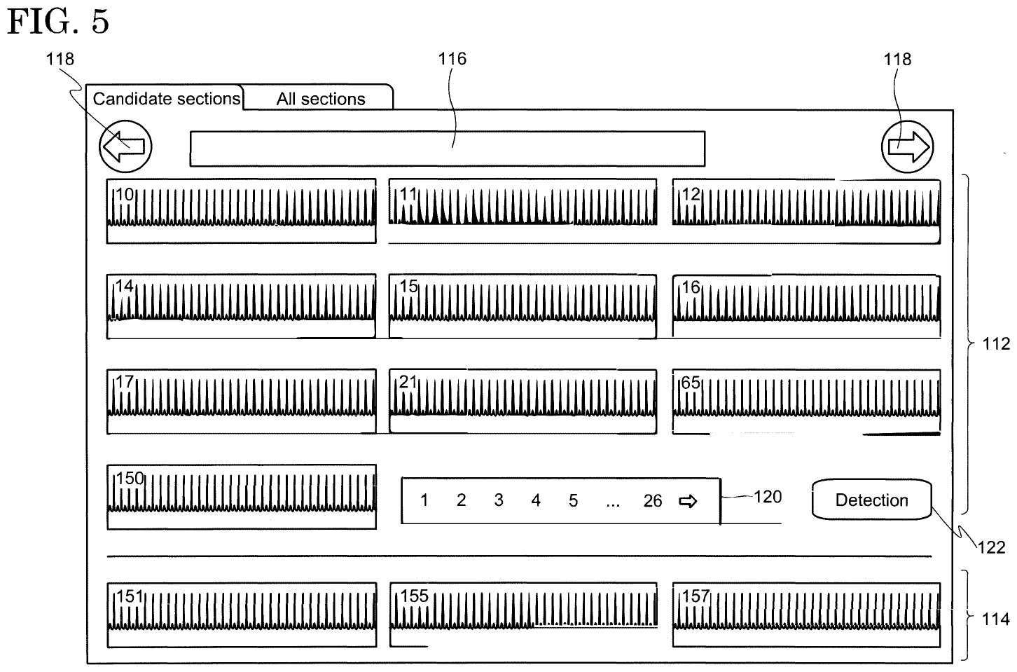

As shown in FIG. 4, the identifier or its corresponding number provided to each section may be displayed on or near each section. A plurality of candidate sections (in this case, 11^{th} to 13^{th} sections) following this plurality of candidate sections may be further displayed in a lower region 114 in chronological order on the screen. In addition, a region 116 may be provided to indicate the subject's attributes (name, age, gender, date and time of electrocardiogram acquisition, subject's identification information, and the like). In addition, an icon 118 or the like may be placed on the screen to accept an input for displaying subsequent or preceding candidate sections which are not displayed on the screen. When the candidate sections are displayed over a plurality of pages, a region 120 may be further provided for accepting an input to directly transfer to an arbitrarily selected page. All of the candidate sections from the first section to the 13^{th} section are shown in the example shown in FIG. 4. However, the specified sections are not selected as the candidate sections. Therefore, all of the consecutive sections are not always shown on one screen, and the numbers of adjacent candidate sections may not be consecutive as shown in FIG. 5. As described in detail below, an icon 122 is provided on the screen for accepting user commands to cause the electrocardiogram analyzer 140 to execute the detection of disease or test abnormalities.

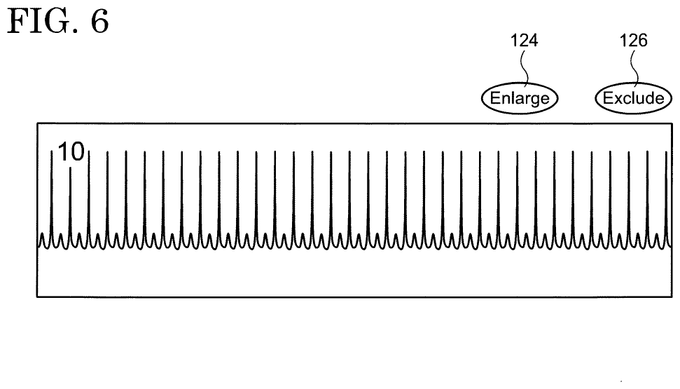

An enlarged view of one candidate section is shown in FIG. 6. As shown in FIG. 6, an icon 124 for accepting a user command to enlarge the candidate section or an icon 126 for accepting a user command to eliminate the candidate section as the non-analysis section may be placed on or near each candidate section. When the user inputs a user command via the icon 124, the support device 110 expands the candidate section on the screen of the display device.

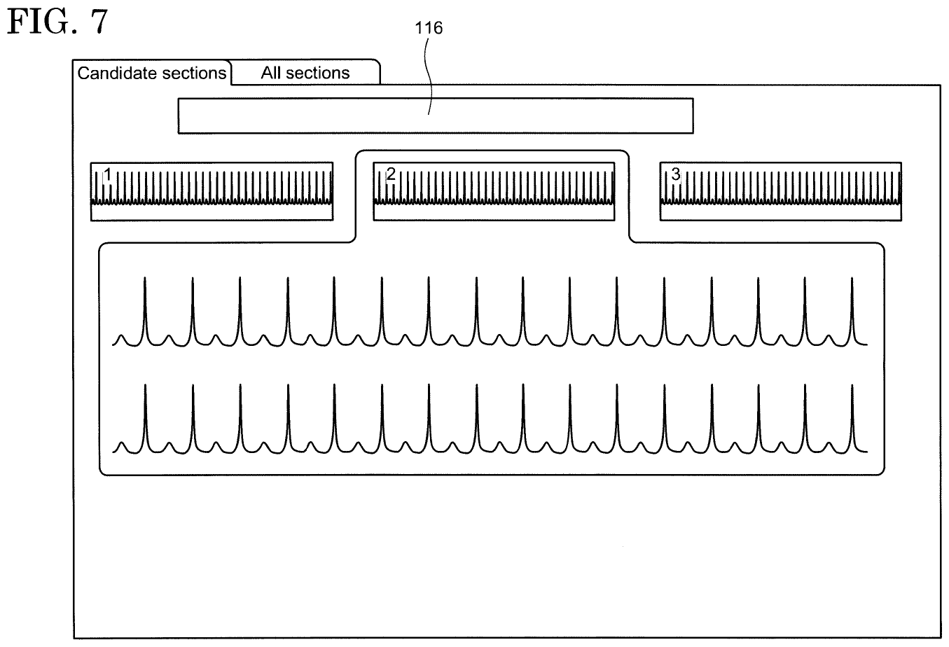

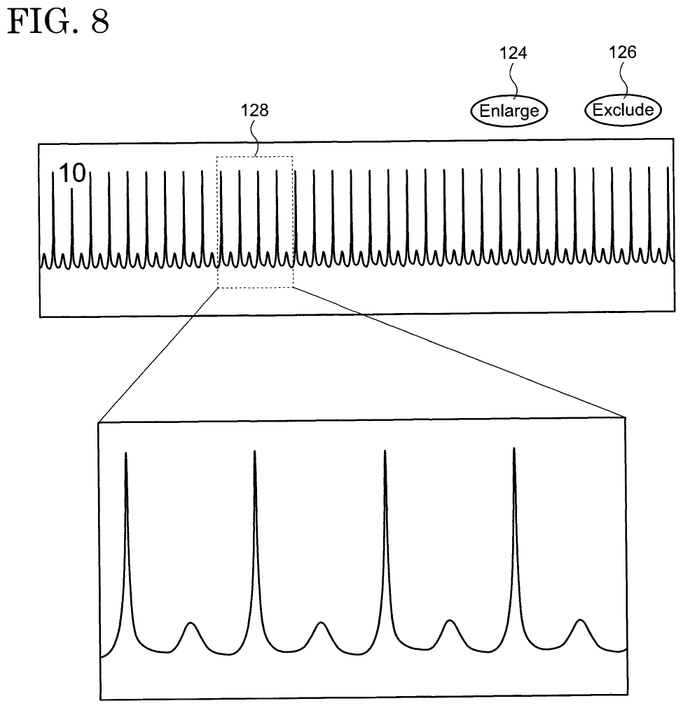

The method of enlargement at this time may also be set arbitrarily. For example, the candidate section to be enlarged may be enlarged over one or a plurality of lines so as to overlap with other candidate sections which are not enlarged as shown in FIG. 7. Alternatively, the user may select a region 128 to be enlarged on the screen to enlarge this region as shown in FIG. 8. The user can observe each candidate section in more detail and determine whether or not this candidate section is suitable for the secondary analysis on the electrocardiogram analyzer 140 by enlarging and displaying each candidate region in this manner.

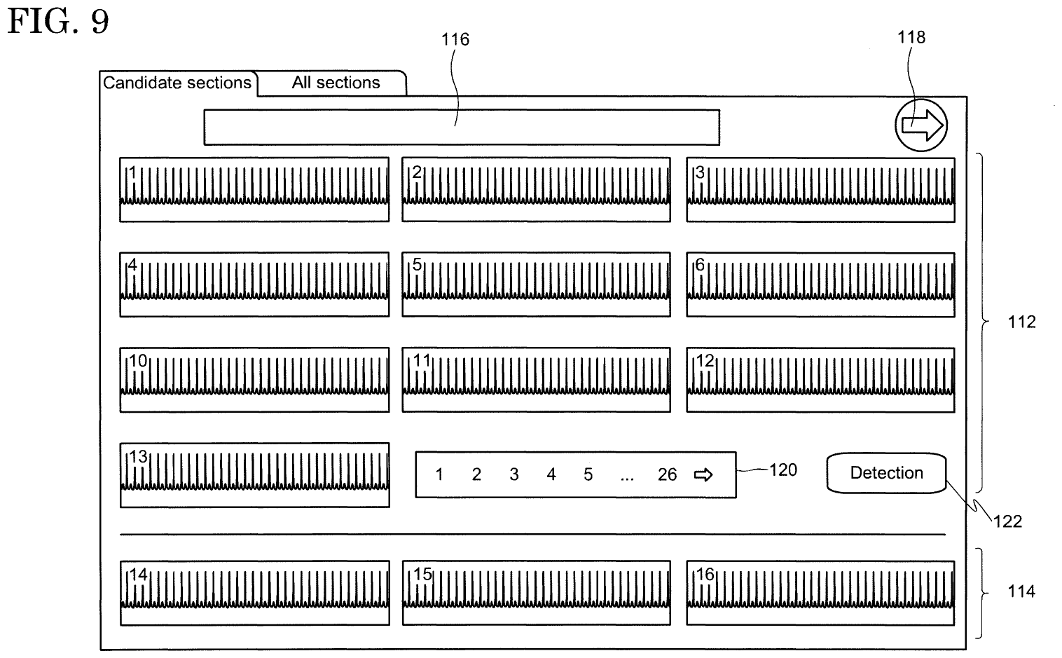

When the user determines that a candidate section is not suitable for the secondary analysis by the electrocardiogram analyzer 140, the user selects it as the non-analysis section and excludes it from the candidate sections using the icon 126 (see FIG. 6). Upon receiving the input to the icon 126, the support device 110 erases this candidate section from the screen and displays the subsequent candidate section in accordance with the program's instructions. For example, when the seventh section to ninth section among the candidate sections shown in FIG. 4 are excluded, these candidate sections are erased from the screen, and the subsequent sections (the 11^{th} section to the 13^{th} section) are displayed in the upper region 112 in chronological order (see FIG. 9). In addition, the sections (the 14^{th} section to the 16^{th} section) following in chronological order are displayed in the lower area 114. This manner allows the user to observe the candidate sections in a short time without any stress. At the same time, the support device 110 transmits the identifier of the section selected as the non-analysis section to the electrocardiogram analyzer 140. The electrocardiogram analyzer 140 flags the identifier of the section selected as the non-analysis section to indicate that it was excluded from the candidate sections by the user. On the other hand, the candidate sections which are not selected by the user remain as the analysis sections. The candidate sections used for the secondary analysis are selected by the above process.

The number of candidate sections selected as the non-analysis sections is generally far fewer than the number of candidate sections which remain as the analysis sections. Therefore, the operation to select the non-analysis sections from the candidate sections is extremely less burdensome for the user compared with the operation to select the analysis sections from the candidate sections displayed on the screen. Therefore, it is possible to quickly select and collect the analysis sections without placing a heavy burden on the user by allowing the user to select the non-analysis sections from the candidate sections.

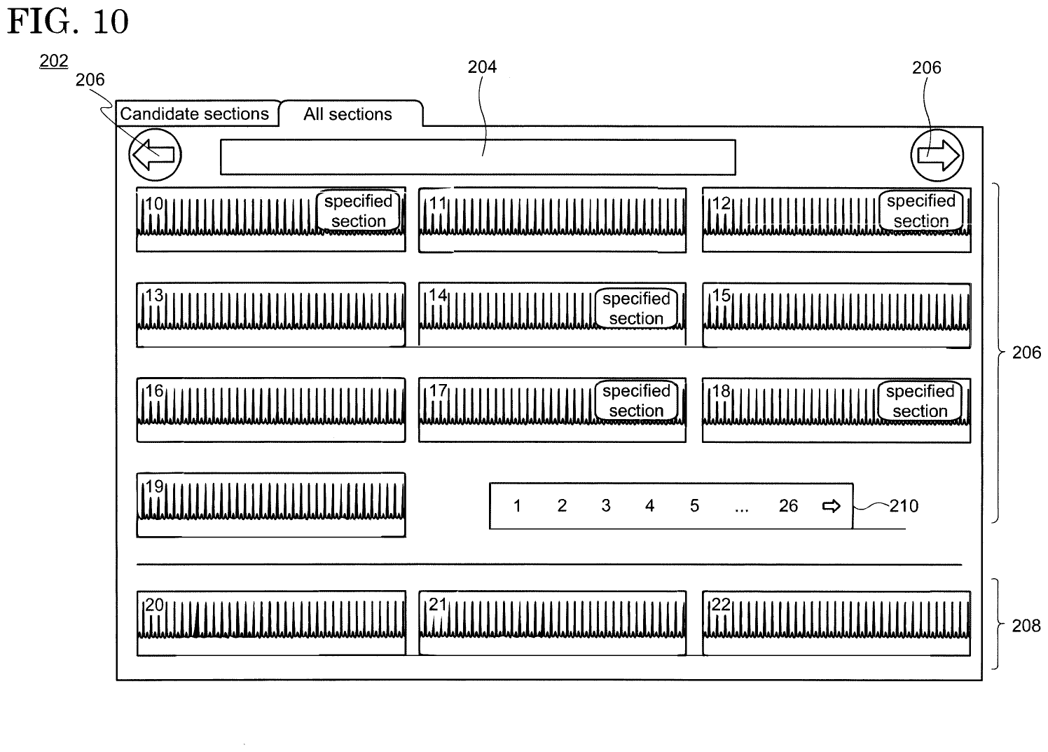

Note that the support device 110 may be configured to display all of the sections on the screen of the display device according to the instructions of the program as an optional display method. The display method can also be set arbitrarily in this case. For example, a tab different from a tab displaying the candidate sections may be provided, and all of the sections may be displayed on this tab as shown in FIG. 10. At this time, an indication may be placed on or near each section to distinguish between the sections extracted as the candidate sections and the specified sections excluded by the primary analysis. This method provides the user with an opportunity to verify the judgment results of the primary analysis so that the user can determine whether or not the section suitable for the secondary analysis was mistakenly excluded in the primary analysis.

Furthermore, the support device 110 may be configured to display the plurality of candidate sections on the screen in chronological order from the candidate section arbitrarily selected by the user as an optional display method.

For example, the user may select the candidate section to be displayed at the top in consideration of the subject's conditions. For example, the candidate section to be displayed at the top may be selected on the basis of the information acquired by the heart rate monitor 170 and the accelerometer 180. Specifically, one of the candidate sections acquired when the subject is asleep, at rest, or exercising may be displayed at the top, and subsequent candidate sections may be displayed from this candidate section in chronological order.

### (4) Secondary Analysis

The secondary analysis is then performed using the analysis sections. That is, when the selection of the non-analysis sections is completed, the user manipulates the icon 122. Upon acceptance of the user command via the icon 122, the support device 110 transmits an instruction to the electrocardiogram analyzer 140 to execute detection of disease or test abnormalities using the analysis sections. The electrocardiogram analyzer 140 follows this command and executes the detection of disease or test abnormalities. In the secondary analysis, a variety of machine learning algorithms described for the primary analysis can be used. The output data are the judgment results for each section and include two categories: whether or not each section is a section in which the diseases or the test abnormalities listed by the user as test items appear. Similar to the primary analysis, the secondary analysis may be performed for each analysis section, for all of the analysis sections simultaneously, or for multiple analysis sections (e.g., 10 analysis sections).

### (5) Display of Analysis Results

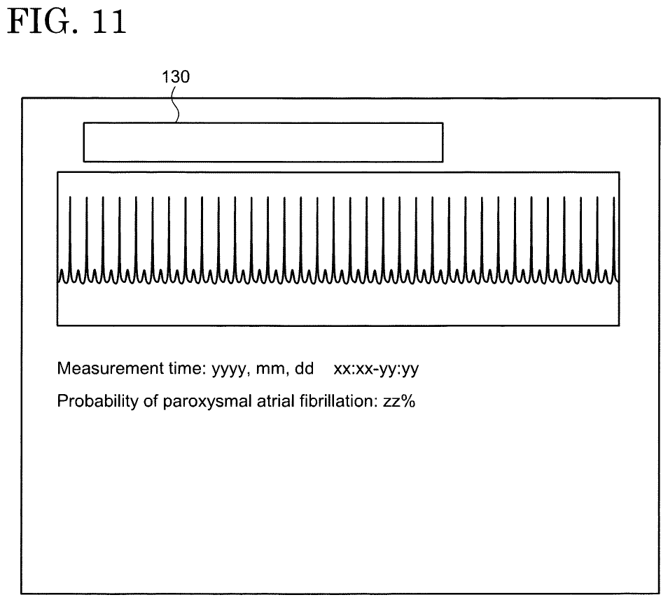

The results of the secondary analysis are displayed on the screen of the display device of the support device 110. The method of display at this time may also be arbitrarily set. For example, the entire electrocardiogram or a portion of the electrocardiogram may be displayed in addition to a region 130 for indicating the attributes of the subject as shown in FIG. 11. For example, a section showing waveforms suggesting a disease or test abnormality may be displayed. In addition, various information to assist the user's medical treatment, such as the date and time of the electrocardiogram measurement, the date and time of the electrocardiogram analysis, and the probability, degree, or score of the disease or the test abnormality, may be displayed. The past electrocardiogram analysis results of the subject may also be further displayed.

Note that, although the user inputs user commands by operating a variety of icons in the electrocardiogram analysis described above, there are no restrictions on the method of inputting user commands. The user may select user commands from pull-down menus or may input user commands by a keyboard operation or voice. Furthermore, the aforementioned primary analysis and secondary analysis may be performed with the same electrocardiogram analyzer or with different electrocardiogram analyzers.

As described above, in the electrocardiogram analysis using the present support system 100, the electrocardiogram is divided into a plurality of sections by the electrocardiogram analyzer 140, and a part of the plurality of sections is extracted as the candidate sections appropriate for the secondary analysis in the pretreatment after the primary analysis. Furthermore, the candidate sections are provided for verification by the user, which allows only the candidate sections appropriate for detection of various diseases and test abnormalities by the electrocardiogram analyzer 140 to be selected as analysis sections. Therefore, in the secondary analysis conducted by the electrocardiogram analyzer 140, the electrocardiogram analysis can be performed using the analysis sections which have less noise and which are appropriate for the detection of diseases and test abnormalities. As a result, various diseases, their signs, and test abnormalities can be detected more precisely.

### 3. Program

An embodiment of the present invention is a program for supporting the electrocardiogram analysis performed in the electrocardiogram analyzer 140 using the support device 110. An embodiment of the invention is also a computer-readable storage medium in which this program is recorded. This program may be installed in the support device 110 or may be a program configured to be run by a user on a network using a browser installed in the support device 110.

The program is configured to cause the computing device of the support device 110 to execute the instructions for the aforementioned electrocardiogram analysis. Accordingly, the program is configured to accept the user command to upload the subject's electrocardiogram to the electrocardiogram analyzer 140. Upon accepting this user command, the program causes the computing device to execute the uploading of the electrocardiogram stored in the support device 110 or the database 160 to the electrocardiogram analyzer 140 via the network 102.

This program further causes the computing device to display all of the sections of the electrocardiogram or the plurality of candidate sections extracted by the electrocardiogram analyzer 140 on the screen of the display device of the supporting device 110. The program may be configured to transmit all of the plurality of sections structuring the electrocardiogram or the candidate sections to the support device 110. The program is configured to cause the supporting device 110 to accept the user command to enlarge the candidate sections, the user command to select the candidate section as the non-analysis section, and the user command to cause the electrocardiogram analyzer 140 to perform detection of disease or test abnormalities using the analyzed sections. When these user commands are accepted, the program causes the computing device of the support device 110 to execute the corresponding operations, i.e., enlargement of the candidate sections, transmission of the identifier of the candidate section selected as the non-analysis section to the electrocardiogram analyzer 140 and deletion of the candidate section selected as the non-analysis section from the screen, and transmission of the instruction to the electrocardiogram analyzer 140 to analyze the electrocardiogram using the analysis sections to detect diseases and test abnormalities. The program may further be configured to cause the computing device of the support device 110 to accept the user command to display all of the plurality of sections of the electrocardiogram on the screen and to display all of the sections on the screen. At this time, the program may cause the computing device of the support device 110 to execute a display to identify the sections which were not extracted as the candidate sections by the electrocardiogram analyzer 140.

Examples of the aforementioned computer-readable storage medium include a magnetic medium such as a hard disk, a flexible disk, and a magnetic tape, an optical medium such as a CD-ROM and a DVD, a magneto-optical medium such as a floptical disk, and a hardware device configured to store or execute the program such as ROM, RAM, and a flash memory. The instructions of the program include machine language code such as that generated by a compiler as well as high-level language code executed by a server using an interpreter or the like. The program may be configured to be installed from the computer-readable storage medium to the computing device of the support device 110. Alternatively, the program may be downloadable to the computing device via the network 102.

The aforementioned modes described as the embodiments of the present invention can be implemented by appropriately combining with each other as long as no contradiction is caused. Furthermore, any mode which is realized by persons ordinarily skilled in the art through the appropriate addition, deletion, or design change of elements or through the addition, deletion, or condition change of a process on the basis of each embodiment is included in the scope of the present invention as long as they possess the concept of the present invention.

It is understood that another effect different from that provided by each of the aforementioned embodiments is achieved by the present invention if the effect is obvious from the description in the specification or readily conceived by persons ordinarily skilled in the art.

1. A support system for electrocardiogram analysis, the support system comprising:
an electrocardiogram analyzer configured to divide an electrocardiogram of a subject into a plurality of sections that includes specified sections and candidate sections; and
a support device configured to be communicatively connected to the electrocardiogram analyzer,
wherein the support device comprises:
a computing device; and
a display device controlled by the computing device,
wherein the computing device is configured to execute:
accepting a first user command to upload the electrocardiogram to the electrocardiogram analyzer, wherein the electrocardiogram was acquired using an electrocardiograph over a time period that is equal to or longer than 1 hour, and wherein a duration of each section of the plurality of sections of the electrocardiogram is between about 5 seconds and about 120 seconds;
uploading the electrocardiogram to the electrocardiogram analyzer;
displaying the candidate sections on a screen of the display device;
accepting a second user command to select a non-analysis section from the candidate sections, such that the second user command causes the candidate sections to be divided into analysis sections that are not selected by the second user command and non-analysis sections that are selected by the second user command; and
transmitting an instruction to the electrocardiogram analyzer to initiate a secondary analysis to analyze the electrocardiogram using the analysis sections, and
wherein the electrocardiogram analyzer is configured to:
analyze, prior to the secondary analysis, the plurality of sections in a primary analysis using a supervised learning model prepared by machine learning using, as teaching data, electrocardiogram data exhibiting a disease and electrocardiogram data which does not exhibit the disease to identify the specified sections as indicating a waveform caused by the disease;
automatically exclude the specified sections from the plurality of sections; and
simultaneously analyze multiple analysis sections in the secondary analysis using a supervised learning model prepared in advance by machine-learning characteristics of diseases or test abnormalities, which cannot be identified by the user, using, as teaching data, first electrocardiogram data and second electrocardiogram data each of which does not exhibit the disease or the test abnormalities, wherein the first electrocardiogram data is acquired from humans having the disease and the second electrocardiogram data is acquired from humans who do not have the disease.

2. (canceled)

3. (canceled)

4. The support system according to claim 1,
wherein the electrocardiogram analyzer is further configured to judge whether the electrocardiogram data exhibits a sign of the disease or an abnormality suspected of the disease on the basis of the analysis.

5. The support system according to claim 1,
wherein the computing device is configured to display the candidate sections acquired after an arbitrarily selected time point and subsequent candidate sections thereof on the screen in chronological order.

6. The support system according to claim 1,
wherein the computing device is further configured to display all of the plurality of sections on the screen.

7. The support system according to claim 6,
wherein the computing device is further configured to display the specified sections on the screen.

8. The support system according to claim 1,
wherein the computing device is further configured to
display n of the candidate sections on the screen in chronological order,
delete the candidate section selected as the non-analysis section from the screen according to the second user command, and
display one candidate section subsequent to the n of the candidate sections on the screen, and
wherein n is selected from integers equal to or greater than 1 and equal to or less than 20.

9. A support device for electrocardiogram analysis comprising:
a computing device; and
a display device controlled by the computing device,
wherein the computing device is configured to execute:
accepting a first user command to cause the computing device to upload an electrocardiogram of a subject to an electrocardiogram analyzer which is communicatively connected to the computing device, configured to divide an electrocardiogram into a plurality of sections that includes specified sections and candidate sections;
uploading the electrocardiogram to the electrocardiogram analyzer,
displaying the candidate sections on a screen of the display device,
accepting an input of a second user command for selecting a non-analysis section from the candidate sections, such that the second user command causes the candidate sections to be divided into analysis sections that are not selected by the second user command and non-analysis sections that are selected by the second user command, and
transmitting, to the electrocardiogram analyzer, a command to initiate a secondary analysis for analyzing the electrocardiogram of the subject using the analysis sections,
wherein the electrocardiogram analyzer is configured to:
analyze, prior to the secondary analysis, the plurality of sections in a primary analysis using a supervised learning model prepared by machine learning using, as teaching data, electrocardiogram data exhibiting a disease and electrocardiogram data which does not exhibit the disease to identify the specified sections as indicating a waveform caused by the disease;
automatically exclude the specified sections from the plurality of sections; and
simultaneously analyze multiple analysis sections in the secondary analysis using a supervised learning model prepared in advance by machine-learning characteristics of diseases or test abnormalities, which cannot be identified by the user, using, as teaching data, first electrocardiogram data and second electrocardiogram data each of which does not exhibit the disease or the test abnormalities, wherein the first electrocardiogram data is acquired from humans having the disease and the second electrocardiogram data is acquired from humans who do not have the disease.

10. The support device according to claim 9,
wherein the computing device is configured to display the candidate sections acquired after an arbitrarily selected time point and subsequent candidate sections thereof on the screen in chronological order.

11. The support device according to claim 9, further configured to display all of the plurality of sections on the screen.

12. The support device according to claim 11, further configured to perform a display for specifying the specified sections on the screen.

13. The support device according to claim 9,
wherein the computing device is further configured to
display n of the candidate sections on the screen in chronological order,
delete the candidate section selected as the non-analysis section from the screen according to the second user command, and
display one candidate section subsequent to the n of the candidate sections on the screen, and
wherein n is selected from integers equal to or greater than 1 and equal to or less than 20.

14. A program for supporting electrocardiogram analysis performed on an electrocardiogram analyzer using a computing device, the electrocardiogram analyzer being communicatively connected to the computing device and being configured to divide an electrocardiogram of a subject to a plurality of sections the include specified sections and candidate sections, the program being configured to cause the computing device to execute:
accepting a first user command to upload the electrocardiogram of the subject to the electrocardiogram analyzer;
uploading the electrocardiogram to the electrocardiogram analyzer;
displaying the candidate sections on a screen of the display device of the computing device;
accepting a second user command for selecting a non-analysis section from the candidate sections, such that the second user command causes the candidate sections to be divided into analysis sections that are not selected by the second user command and non-analysis sections that are selected by the second-user command;
transmitting, to the electrocardiogram analyzer, a command to initiate a secondary analysis for analyzing the electrocardiogram using the analysis sections; and
analyzing the electrocardiogram using the analysis sections in the secondary analysis using a supervised learning model prepared by machine learning,
wherein the electrocardiogram analyzer is configured to:
analyze, prior to the secondary analysis, the plurality of sections in a primary analysis using a supervised learning model prepared by machine learning using, as teaching data, electrocardiogram data exhibiting a disease and electrocardiogram data which does not exhibit the disease to identify the specified sections as indicating a waveform caused by the disease;
automatically exclude the specified sections from the plurality of sections; and
simultaneously analyze multiple analysis sections in the secondary analysis using a supervised learning model prepared in advance by machine-learning characteristics of diseases or test abnormalities, which cannot be identified by the user, using, as teaching data, first electrocardiogram data and second electrocardiogram data each of which does not exhibit the disease or the test abnormalities, wherein the first electrocardiogram data is acquired from humans having the disease and the second electrocardiogram data is acquired from humans who do not have the disease.

15. The program according to claim 14, further configured to cause the computing device to execute displaying the candidate sections acquired after an arbitrarily selected time point and subsequent candidate sections thereof on the screen in chronological order.

16. The program according to claim 14, further configured to cause the computing device to execute displaying all of the plurality of sections on the screen.

17. The program according to claim 16, further configured to cause the computing device to execute performing a display for specifying the specified sections on the screen.

18. The program according to claim 14, further configured to cause the computing device to execute:
displaying n of the candidate sections on the screen in chronological order,
deleting the candidate section selected as the non-analysis section from the screen according to the second user command, and
displaying one candidate section subsequent to the n of the candidate sections on the screen,
wherein n is selected from integers equal to or greater than 1 and equal to or less than 20.
